(19) United States
(12) Plant Patent Application Publication
(10) Pub. No.: US 2018/0132400 P1
(43) Pub. Date: May 10, 2018

(54) ILLICIUM PLANT NAMED 'NCIH2'

(50) ;

(71) Applicant: North Carolina State University, Raleigh, NC (US)

(72) Inventor: Thomas G. Ranney, Raleigh, NC (US)

(21) Appl. No.: 15/330,892

(22) Filed: Nov. 10, 2016

(51) Int. Cl. A01H 5/02

(52) U.S. Cl. PLT/226

(57) ABSTRACT

‘NCIH2’ is a new cultivar of hybrid Illicium with remontant flowering, a compact habit, and large, white flowers.

Latin name of the genus and species: The Latin name of the novel plant variety disclosed herein is Illicium.

Variety denomination: The inventive hybrid of Illicium disclosed herein has been given the varietal denomination ‘NCIH2’.

### BACKGROUND OF THE INVENTION

The present invention comprises a new and distinct variety of Illicium, hereinafter referred to by the cultivar name ‘NCIH2’. ‘NCIH2’ was selected from a population of seedlings grown from an open-pollinated F₁ hybrid of Illicium mexicanum×Illicium floridanum (2014-318, unpatented). The first asexual propagation of ‘NCIH2’ was carried out in July 2007 by rooting stem cuttings in Mills River, N.C. and has been asexually reproduced repeatedly by vegetative cuttings over a 9-year period. ‘NCIH2’ roots readily from softwood cuttings treated with a basal dip of 5,000-10,000 ppm indole butyric acid (potassium salt) in water. ‘NCIH2’ has been found to retain its distinctive characteristics through successive asexual propagations.

### SUMMARY OF THE INVENTION

The following are the unique combination of characteristics of this new cultivar when grown under standard horticultural practices in Mills River, N.C.

1. Remontant flowering: Primarily in the spring and fall and sporadically throughout the growing season.

2. A compact, upright habit.

3. Large, white flowers.

### BRIEF DESCRIPTION OF THE DRAWINGS

This new Illicium is illustrated by the accompanying photographs which show the plant's flowers, foliage and form. The colors shown are as true as can be reasonably obtained by conventional photographic procedures. Colors in the photographs may differ slightly from the color values cited in the detailed botanical description, which accurately describe the colors of the new hybrid Illicium.

FIG. 1 is a photograph showing the habit and fall flowering of ‘NCIH2’ September 2009 on a four-year-old field-grown shrub.

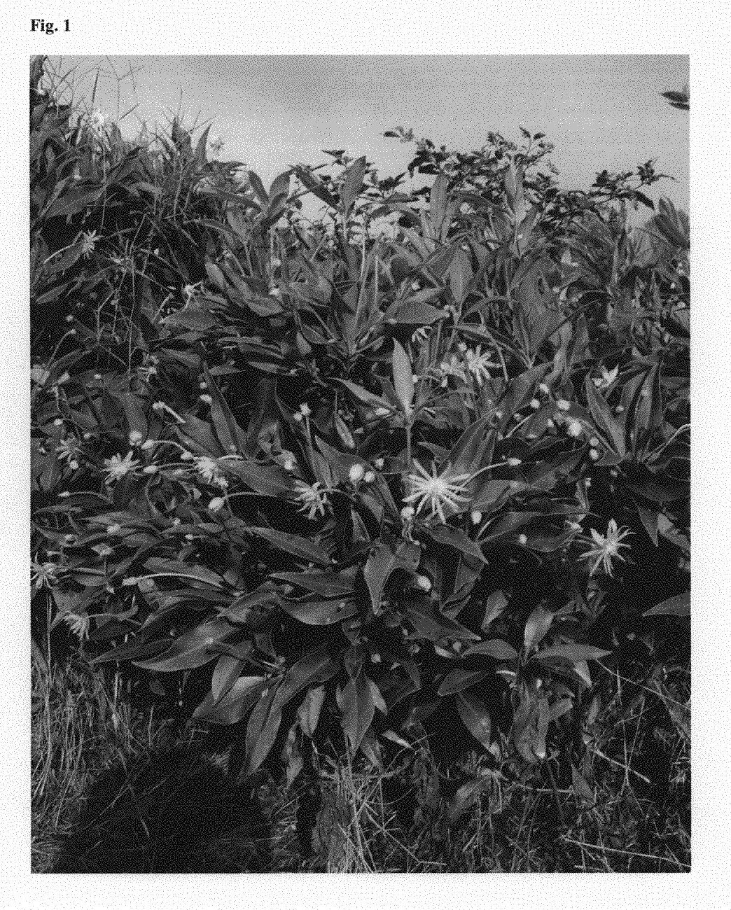

FIG. 2 is a photograph showing the flower of ‘NCIH2’ in spring 2013 on a three-year-old container-grown shrub.

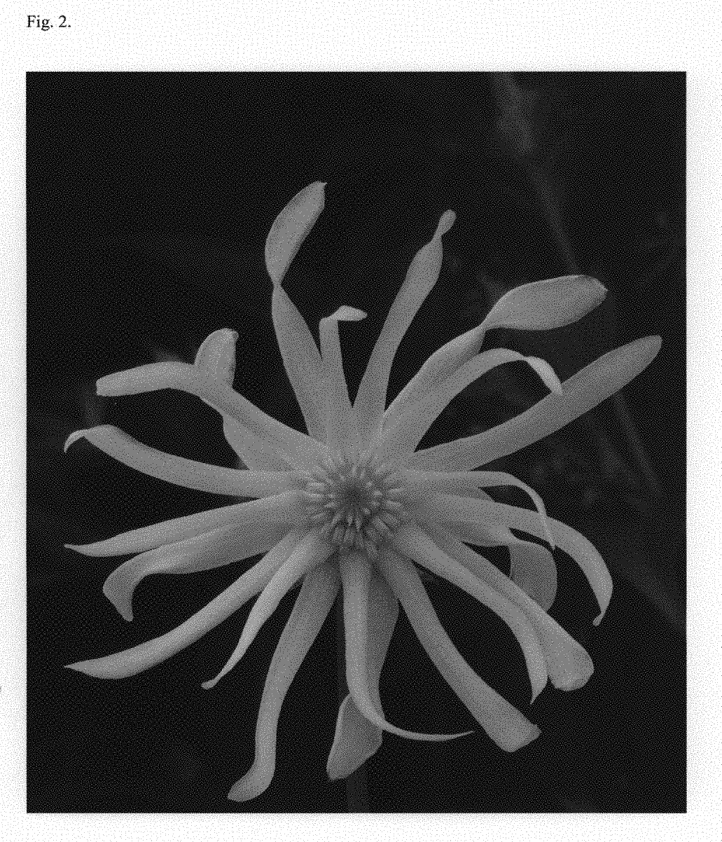

FIG. 3 is a photograph showing the flower morphology of ‘NCIH2’ in July 2013 on a three-year-old container-grown shrub.

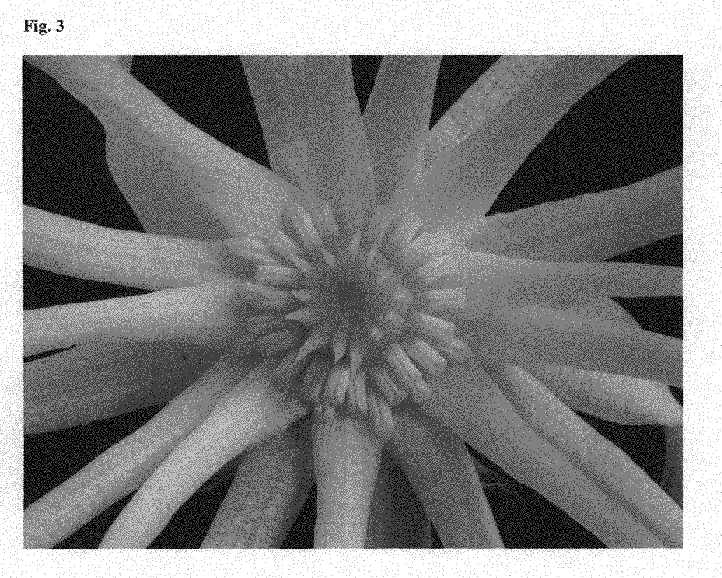

### DETAILED BOTANICAL DESCRIPTION

The following is a detailed description of the botanical characteristics of the new and distinct hybrid Illicium variety plant known by the denomination ‘NCIH2’. The detailed description was taken on a three-year-old field-grown plant in Mills River, N.C. in 2015. All colors cited herein refer to The Royal Horticultural Society (R.H.S.) Colour Chart, London, sixth edition, 2015. Where specific dimensions, sizes, colors, and other characteristics are given, it is to be understood that such characteristics are approximations or averages set forth as accurately as practicable.

### TECHNICAL DESCRIPTION OF THE VARIETY

Classification:
   Botanical name.—Illicium ‘NCIH2’.
   Common name.—Anise-tree.
Parentage: Seedling grown from an open-pollinated, F₁ hybrid Illicium mexicanum×Illicium floridanum (2014-318, unpatented)).
Plant description:
   Growth habit.—Upright, compact shrub.
   Height.—Approximately 1.5 m at maturity.
   Width.—Approximately 1 m at maturity.
Shoot and stem:
   Lateral branch length.—Avg. 29 cm (range 22 to 46 cm).
   Lateral branch diameter.—Avg. 0.45 cm (0.3 to 0.6 cm).
   Internode length.—Avg. 9.9 cm (7 to 12 cm).
   Texture.—Glabrous.
   Immature shoot color.—Yellow-Green (146B).
   Mature stem color.—Grey-Brown (199A).
Leaves:
   Type.—Simple.
   Persistence.—Evergreen.
   Arrangement.—Alternate to pseudo-whorled.
   Shape.—Elliptical.
   Apex.—Acuminate.
   Base.—Cuneate.
   Venation.—Reticulate.
   Vein color.—Upper: Yellow-Green (144B). Lower: Yellow-Green (144B).
   Margins.—Entire.
   Emerging leaves.—Color: Upper: Yellow-Green (144A). Lower: Yellow-Green (144A). Texture: Glabrous, shiny on both upper and lower emerging leaves.
   Mature leaves during growing season.—Color: Upper: Yellow-Green (146A). Lower: Yellow-Green (146C). Texture: Glabrous, leathery both upper and lower mature leaves. Length of lamina: Avg. 12.3 cm (9.3 to 17.1 cm). Width: Avg. 3.2 cm (2.4 to 4.3 cm). Foliage scent: Aromatic, anise-like. Leaf attachment: Petiolate.
   Petiole.—Length: Avg. 1.3 cm (0.6 to 1.6 cm). Diameter: Avg. 0.2 cm (0.15 to 0.3 cm). Color: Yellow-Green (144B). Texture: Glabrous.
Inflorescence:
   Flowering season.—Two primary blooming periods, in April-May and again in September-November in Mills River, N.C. Flowers are also produced sporadically throughout the growing season.
   Arrangement.—Single flowers, occurring at leaf axils, 1-11 flowers per axil.
   Fragrance.—Slight anise fragrance.
   Bud.—Shape: Oblong. Length: Avg. 1.5 cm (1.2 to 1.9 cm). Diameter: Avg. 0.8 cm (0.7 to 1 cm). Color: Yellow-Green (145B).
   Perianth.—Diameter: Avg. 5.4 cm (5 to 6.5 cm). Height: Avg. 1.2 cm (1 to 1.4 cm).
   Perianth aspect.—Outward facing to slightly nodding.
   Petals.—Arrangement: Radial, slightly overlapping. Number: Avg. 22 (18 to 26 petals). Texture: Smooth. Shape: Strap-shaped. Margin: Entire. Apex: Acute to acuminate. Length: Avg. 2.2 cm (1.2 to 2.7 cm). Width: Avg. 0.25 cm (0.15 to 0.4 cm). Color when opening: Upper: Yellowish-White (NN155A). Lower: Yellowish-White (NN155A). Color at maturity: Upper: Yellowish-White (NN155A). Lower: Yellowish-White (NN155A).
   Sepal.—Shape: Oblong. Number: Avg. 3 (2-6 sepals). Color, immature: Upper: Yellowish-White (NN155A). Lower: Yellowish-White (NN155A). Color, mature: Upper: Yellowish-White (NN155A). Lower: Yellowish-White (NN155A). Texture: Glabrous. Length: Avg. 1.8 cm (1.2 to 2.4 cm). Width: Avg. 0.6 cm (0.5 to 0.6 cm).
   Peduncle.—Length: Avg. 3.3 cm (2.2 to 4.5 cm). Diameter: Avg. 0.18 cm (0.15 to 0.2 cm). Color: Yellow-Green (144C). Surface texture: Glabrous.
Reproductive organs:
   Gynoecium.—Pistil number: 1 aggregate of 9-14 pistils. Pistil length: 0.5 cm. Style length: 0.2 cm. Style color: Yellow-Green (145C).
   Androecium.—Stamen number: 12 to 36. Stamen length: 0.2 cm. Stamen width: 0.1 cm. Stamen color: Green-White (157A).
Fruit and seed:
   Fruit type.—Star-shaped, woody follicetum.
   Fruit diameter.—Avg. 2.3 cm (2.0 to 2.8 cm).
   Fruit color.—Immature: Yellow-Green (N144A). Mature: Brown (N200A).
   Seed number.—Avg. 13 (9 to 14) per fruit.
Disease and insect resistance: No significant disease or insect pests have been observed.
Cold hardiness: At least USDA zone 6a; testing has not been completed in colder zones.

### COMPARISON WITH COMMERCIAL CULTIVARS

Table 1 shows distinguishing characteristics between ‘NCIH2’ and other cultivars. ‘NCIH2’ is distinguished from Illicium ‘Woodland Ruby’ by having a more compact habit, white flowers, and more remontant flowering. ‘NCIH2’ is distinguished from Illicium ‘NCIH1’ in that ‘NCIH2’ has white flowers and ‘NCIH1’ has red flowers.

TABLE 1
Comparison to commercial cultivars.

| | Taxa | | |
|---|---|---|---|
| | Illicium | Illicium | Illicium |
| Trait | ‘Woodland Ruby’ | ‘NCIH1’ | ‘NCIH2’ |
| Flowering season | Mostly | Remontant. Mostly | Remontant. Mostly |
| | April-May | in April-May and | in April-May and |
| | | September-November | September-November |
| Plant habit and size | Upright to 2 m tall | Upright to 1.5 m tall | Upright to 1.5 m tall |
| | and 1.5 m wide | and 1 m wide | and 1 m wide |
| Branch length | Avg. 62 cm | Avg. 30 cm | Avg. 29 cm |
| Petal color | Purplish Pink | Red-purple | Yellowish-White |
| | (RHS 63C to 67C) | (RHS 60A) | (RHS NN155A) |
| Leaf length | Avg. 10.6 cm | Avg. 9.9 cm | Avg. 12.3 cm |

What is claimed:

1. A new and distinct hybrid Illicium plant named ‘NCIH2’ substantially as illustrated and described herein.
